(19) United States
(12) Patent Application Publication
(10) Pub. No.: US 2016/0368642 A1
(43) Pub. Date: Dec. 22, 2016

(54) METHOD FOR INSPECTING PACKAGING CONTAINER CONTENT

(71) Applicant: Noriyuki INOUE, Kanagawa (JP)

(72) Inventor: Noriyuki INOUE, Kanagawa (JP)

(21) Appl. No.: 14/901,771

(22) PCT Filed: Jun. 26, 2014
(86) PCT No.: PCT/JP2014/067042

§ 371 Date: Dec. 29, 2015

(30) Foreign Application Priority Data
Jul. 4, 2013 (JP) 2013-153048

(51) Int. Cl. B65B 57/10; G01N 33/15; A61J 1/03; G01N 25/72; B65D 25/04; B65D 75/36

(52) U.S. Cl. CPC B65B 57/10; B65D 25/04; B65D 75/366; A61J 1/035; G01N 25/72; G01N 33/15

(57) ABSTRACT

An inspection method is provided to inspect whether abnormality such as omission, chip, abnormal size, abnormal shape, and contamination exists or not in a content that is covered with a packaging material, especially an opaque packaging material. Further, an inspection method of a content in a packaging container is provided with which inspection of a content can be performed rapidly, accurate inspection is possible for any combination of a content and a packaging material, there is no adverse effect on a content such as a pharmaceutical agent or food, no reputational damage is caused, and an inspection apparatus can be installed easily in existing manufacturing equipment. These are performed by an inspection method in which inspection to detect abnormality in the content is performed employing either a difference in increasing speed of temperature or a difference in decreasing speed of temperature of the packaging container.

### TECHNICAL FIELD

The present invention relates to an inspection method of a content covered with a packaging material.

### BACKGROUND

Inspection of a content that is covered with a transparent packaging material is often performed by means of an inspection method where an optical image is taken by a camera. To inspect whether the content is normal or abnormal by using an optical image, a method comparing two-dimensional image information is generally employed, but there are some problems such that a lot of time is needed for the inspection and a colored packaging material has to be used since identification by an image becomes difficult depending on a combination of materials and colors of the content and the packaging material. In addition, since identification of the content is sometimes impossible due to reflected light, stray light or the like at the time of shooting by a camera, an installation location for the camera is limited, and further, illumination is necessary so that additional equipment is required for manufacturing equipment, and moreover, quality of the content is deteriorated by illumination. It is also a problem that the inspection method using an optical image cannot be employed to inspect a content covered with an opaque packaging material.

In recent years, from the point of enhancing quality assurance, in order to prevent quality deterioration of a content (pharmaceutical agent, food or the like) caused by moisture, air, organic compound gas, ultraviolet light or visible light, use of an opaque packaging material is getting more frequent such as a packaging material made of a synthetic resin film to which a pigment or a dye is added, a metal foil, and a metal thin film such as a metal evaporated film. As an inspection method of a content in a packaging container using an opaque packaging material, X-ray inspection method has been proposed (Patent Document 1). However, there are several problems such that X-ray breaks chemical bonds of ingredients of pharmaceutical agents and food and thus has an adverse effect on their quality, people tend to avoid food or the like irradiated with X-ray, which may cause a so-called reputational damage, and further, an X-ray apparatus is large and requires protection of workers.

It may be possible to inspect by taking an image of a content after the content is placed in a packaging container but before the content is covered with an opaque packaging material, however, process abnormality may arise such that the content spills or a foreign substance is mixed in a period between taking the image and covering with the opaque packaging material, therefore, there is a problem that accurate inspection of the content is not possible.

As an inspection method of a content, there is a method of measuring weight, however, there are problems that, in a case where a plurality of contents are contained in a packaging container (e.g., a blister pack), it is not possible to identify which of the plurality of contents is abnormal so that a discarded amount becomes large, and that effective measures against the cause of abnormality cannot be implemented.

### PRIOR ART DOCUMENTS

### Patent Documents

Patent Document 1: Japanese Unexamined Patent Application Publication No. 2012-225666

### SUMMARY

### Problems to be Solved by the Invention

A method is provided to inspect whether abnormality such as omission, chip, abnormal size, abnormal shape, and contamination exists or not in a content that is covered with a packaging material. Especially, a method is provided to inspect whether abnormality exists or not in a content covered with an opaque packaging material. Further, an inspection method of a content in a packaging container is provided with which inspection of a content can be performed rapidly, accurate inspection is possible for any combination of a content and a packaging material, accurate inspection is possible with no adverse effect on an inspection result from disturbance, an installation location for inspection equipment is not limited, installation of large equipment in manufacturing equipment is not needed, quality of a content is not deteriorated by particular illumination, there is no adverse effect on a content such as a pharmaceutical agent or food, no reputational damage is caused, an inspection apparatus is small-sized, protection of workers is not necessary, and an inspection apparatus can be installed easily in existing manufacturing equipment.

### Means to Solve the Problem

The present invention is as follows.

(1) An inspection method of a content in a packaging container, the inspection method including:

inspecting abnormality in the content by employing either a difference in increasing speed of temperature or a difference in decreasing speed of temperature of the packaging container.

(2) The inspection method of the content in the packaging container of article (1), wherein the packaging container has two or more spaces separated by partitions, a content of a same type is contained in each of the spaces, and temperature of the packaging container corresponding to each of the spaces is measured.

(3) The inspection method of the content in the packaging container of article (2), wherein the packaging container is a blister pack comprising a belt-shaped container film having a pocket formed therein in which the content is contained, and a belt-shaped covering material which is adhered to the container film so as to cover the pocket.

(4) The inspection method of the content in the packaging container of any one of articles (1) to (3), wherein temperature of the packaging container is made uniform before temperature of the packaging container is increased or decreased.

The packaging container of the present invention is a container composed of a packaging material for the purpose of protection of a content, ornament, explanatory description, display, identification and the like. For the packaging material, a synthetic resin film, a natural resin film, paper, a metal thin film, a metal plate and the like or a combination material of these can be used. Further, the packaging material is preferably a synthetic resin film, a natural resin film, a metal foil, a metal thin film such as a metal evaporated film, and a deposition film of a metallic compound such as metal oxide. The synthetic film is preferably polyolefin such as polyethylene and polypropylene, polyvinyl chloride, polyvinylidene chloride, polyethylene terephthalate, polystyrene, polycarbonate, polychlorotrifluoroethylene, polyacrylamide, cyclic polyolefin or the like, and more preferably, polyethylene, polypropylene, polyvinyl chloride, polyvinylidene chloride, polyethylene terephthalate, and polyacrylamide. The metal is most preferably aluminum. The metallic compound deposited on a film used for the packaging material is preferably silicon oxide and titanium oxide. Pigment or dye may be applied to, deposited on or kneaded into the synthetic resin film and the natural resin film. It is preferable that the packaging material is a material having low permeability against ultraviolet light, visible light, moisture and organic compound gas. For these materials for the packaging material, a single material or a packaging material in the form of lamination of two or more materials can be used. In terms of mechanical strength and shielding of hazardous substances, a material in which a synthetic resin film and a metal thin film are laminated is especially preferable. For the covering material of a blister pack, it is preferable to employ a material that is easy to fracture, especially an aluminum foil. In terms of maintenance of quality, an opaque packaging material is preferable, and a packaging material whose transmission coefficient against light of 0.4 to 2.0 μm is 0.4 or less is preferable. This is because when the transmission coefficient of the packaging material is 0.4 or less, deterioration of the content by light can be prevented, which is preferable. Film thickness of the packaging material is preferably 10 to 600 μm, and more preferably 15 to 400 μm. If thickness of the packaging material is too small, rigidity of the packaging material is insufficient and the shape is changed, so that accuracy of measuring surface temperature is lowered, and strength of the packaging material is insufficient and thus, in some cases, damage to the content cannot be prevented, which are not preferable. Further, if thickness of the packaging material is too large, thermal capacity of the packaging material itself is large, so that influence of thermal capacity of the content with respect to a temperature change of the packaging container becomes relatively small, and accordingly, difference between a speed of temperature change of a portion where abnormality such as omission of the content exists and a speed of temperature change of a normal portion becomes small, accuracy of inspection is unfavorably lowered.

The content of the present invention includes, but is not limited to, products such as pharmaceutical agents, food, electric components, machine components, stationery and toys, pharmaceutical agents and food being more preferable and pharmaceutical agents being especially preferable. They may be of any construction and a solid or a jelly-like viscous substance are preferable.

The inspection method of the content of the present invention is a method to inspect whether abnormality such as omission, chip, abnormal size, abnormal shape, contamination and the like exists or not in the content within the packaging container.

Difference in increasing speed of temperature of the packaging container means difference in increasing speed of temperature of each portion of the packaging container in which a content is contained when the packaging container is heated to raise the temperature. Difference in decreasing speed of temperature of the packaging container means difference in decreasing speed of temperature of each portion of the packaging container when cooled. As a method of heating or cooling, such methods are preferable as leaving the packaging container in an atmosphere of a predetermined temperature, blowing air of a predetermined temperature onto the packaging container, and bringing the packaging container into contact with a physical body of a predetermined temperature. The method of blowing air of a predetermined temperature is most preferable, since damage to the packaging container is least as well as temperature can be rapidly changed.

Temperature of the packaging container means either volume temperature or surface temperature. Surface temperature is more preferable since temperature measurement can be done quickly and temperature of a specific portion of the surface of the packaging container can be measured in which a content is contained. While the temperature of the packaging container is increasing or decreasing and before reaching equilibrium temperature, the temperature is measured, and a speed of temperature change is calculated, which is compared with the speed of temperature change of the packaging container having a normal content therein, to inspect whether abnormality of a content is present or not. ‘Before reaching equilibrium temperature’ means a case where the speed of temperature change of the packaging container at a portion having a normal content is 0.2° C./min or more.

As a thermometer to measure temperature of the packaging container of the present invention, any one of common thermometers can be used. As a surface thermometer, a surface thermometer of either a contact type or a noncontact type can be used. Surface thermometers of a contact type include a thermocouple system and a resistance temperature detector system, however, the thermocouple system is preferable since temperature of a minute area can be measured quickly. A noncontact-type surface thermometer is preferable to a contact-type surface thermometer, since the packaging container is hardly damaged and temperature can be measured quickly. As a noncontact-type surface thermometer, an infrared radiation thermometer is preferable. An infrared radiation thermometer of either a thermoelectric sensing system or a photoelectric sensing system is preferable.

As a method to determine an increasing speed or a decreasing speed of temperature of the present invention, temperature of a packaging container is measured twice or more, and the increasing speed or the decreasing speed of the temperature of the packaging container is calculated from a difference of temperature values. Further, in a case where the temperature of the packaging container can be assumed to be stable at a certain temperature due to a treatment for a predetermined period of time or more such as leaving it in an atmosphere of a predetermined temperature, blowing air of a predetermined temperature on it, or bringing it into contact with a physical body of a predetermined temperature, it is possible to omit the first measurement. That is, the changing speed of temperature can be known from a difference between the temperature assumable to be stable and a temperature value of one measurement after the temperature is increased or decreased from that state.

In a case of abnormality such as omission or chip of the content, thermal capacity is smaller than that in a case where the content is normal, so that the speed of temperature increase when heated or the speed of temperature decrease when cooled is higher. In contrast, in a case of abnormality such that a contained amount of a content is more than normal or a foreign substance is mixed, thermal capacity is larger than that in a case where the content is normal, so that the speed of temperature increase when heated or the speed of temperature decrease when cooled is lower. The changing speed of temperature is compared with a changing speed of a portion of the packaging container where the content is normal to determine whether a difference exists or not, and thus, abnormality of the content can be inspected. By employing a one-dimensional array thermal sensor provided in a conveying path of a packaging container, a system where a thermal sensor is moved for scanning, or a surface thermometer sensor of two-dimensional arrangement to obtain a changing speed of temperature in a two-dimensional thermal image, abnormality can be inspected such as difference in a shape of the content, difference in a size of the content, and presence of a foreign substance.

Partitioned spaces of a packaging container of the present invention means spaces which are surrounded such that the content of each space does not cross over a partition between that space and an adjacent space resulting in displacement, exchange or mixing of the content. The partition may be of any type. For example, a packaging material may be adhered at a periphery of a portion covering the content to form a partition, and a material of a certain shape may be placed on an inner surface of the packaging container to form a partition. It is preferable that the individual spaces have a same shape and a same size.

Area of the space is preferably 0.15 to 300 cm² and more preferably 0.3 to 100 cm². The reason for this is as follows: if the area of the space is too small, a surface area of the packaging container corresponding to the space is small, so that measuring accurately surface temperature corresponding to the individual space is unfavorably difficult. If the area of the space is too large, temperature of atmosphere experienced by the individual spaces tends to be different, so that the changing speed of temperature of the packaging container is sometimes varied due to a cause other than the factor that the content is normal or abnormal. Therefore, inspecting whether abnormality of the content exists or not based on a changing speed of temperature is unfavorably difficult.

Temperature of the packaging container corresponding to the individual space of the present invention means either a volume temperature of the individual space of the packaging container or a surface temperature of a surface of the packaging container corresponding to a wall surface of the individual space.

The fact that the contents are of a same type in the present invention means that at least thermal capacity is same.

A perspective view of a blister pack (including a press through pack (PTP)) of the present invention is shown in FIG. 3 and a cross sectional view of a pocket of the blister pack where a tablet is contained is shown in FIG. 4. The blister pack 1 is a packaging container composed of a container film 2 provided with a plurality of pockets 3 and a covering material 5 attached to the container film 2 so as to cover the pockets 3. A tablet 4 is contained in each of the pockets 3; the number of tablet in each pocket may be one or two or more.

The pocket is an example of the partitioned space of the packaging container. The long blister pack 1 of a belt shape shown in FIG. 3, which is formed of a long container film 2 and a long covering material 5, is cut into a plurality of sheets each having, for example, about ten pockets, yielding commercial products. For example, one tablet, capsule, ampule or highly viscous jellylike substance of pharmaceutical agents, food and the like is contained in each of the pockets of the packaging container. Such a container that when pushed at a convex portion of the pocket by a finger, the packaging material (covering material) on the opposite side is broken so that the content can be taken out is also preferable (sometimes referred to as a press through pack). A size of the pocket is preferably 0.15 to 100 cm², and more preferably 0.30 to 30 cm². A film thickness of the container film is preferably 80 to 400 μm, and more preferably 150 to 350 μm. An film thickness of the covering material is preferably 10 to 70 μm, and more preferably 20 to 50 μm. The container film has a main function of protecting the contents, and thus, the packaging material of the container film should be high in mechanical strength. If the film thickness of the container film is too small, mechanical strength of the container film is insufficient not only to fully protect the contents but also to maintain the shape, making accurate measurement of surface temperature of the packaging container unfavorably difficult. If the film thickness of the container film is too large, thermal capacity of the container film itself becomes large, which makes a proportion of thermal capacity of the content in thermal capacity of the packaging container small, so that a difference in changing speed of temperature of the packaging container becomes small between when abnormality exists in the content and when not, unfavorably lowering the accuracy of inspection. Although the covering material should, of course, have a function of protecting the contents, it is preferable that when it is pushed at the side of the container film, the covering material is broken to allow the content to be taken out, so that a film thickness of the covering material should be small to a certain extent. It is preferable that the function of protecting the contents is undertaken mainly by the container film and that the covering material is as thin as possible. If a film thickness of the covering material is too small, protection of the content is insufficient, and deformation is caused making accurate measurement of surface temperature of the packaging container impossible, which is unfavorable. Further, if a film thickness of the covering material is too large, not only taking out the content is difficult but also a proportion of thermal capacity of the packaging material in thermal capacity of the packaging container becomes large, so that difference in changing speed of temperature of the packaging container becomes small between when abnormality exists in the content and when not, unfavorably lowering the accuracy of inspection. When each of the container film and the covering material has a thickness value in the range noted above, surface temperature can be measured accurately so that it is possible to inspect effectively whether abnormality of the content exists or not.

An example of an inspection method of manufacturing a blister pack of the present invention will be described with reference to FIG. 1. In a long container film 2 of a belt shape, pockets 3 are formed by a heating-sucking processing machine 10, the container film 2 is conveyed with openings of the pocked portions 3 being directed upward, tablets 4 of pharmaceutical agents, food and the like are fed to the container film 2 from a tablet-feeding hopper 11 by means of scattering or the like so as to be contained in the pocket 3, a covering material 5 is laid on an inner-side surface of the container film 2 so as to cover the openings of the pockets 3, and the container film 2 and the covering material 5 are adhered to each other with an adhesive agent. The blister pack 1 is introduced in a temperature-uniformizing section 12 through an entrance at the top-left thereof, then redirected by 90 degrees by a roller located just after the entrance and conveyed downward, inverted by a roller located at the bottom and conveyed upward, and discharged through an exit located at the top-right. In this period surface temperature becomes uniform. An image of the blister pack 1 is taken by a succeeding camera 13 for positional information, a print pattern of the pockets is obtained using the image, and infrared emittance of the surface of the packaging container corresponding to the print pattern can be obtained. Temperature is increased or decreased by a succeeding temperature-varying section 14. Again in the temperature-varying section 14, similarly to temperature-uniformizing section 12, the blister pack is redirected by 90 degrees at an entrance located at the top-left and conveyed downward, inverted by a roller at the bottom and introduced, through an exit at the top-right, in a succeeding temperature-measuring section 15. In the temperature-measuring section 15, surface temperature is measured by a thermometer to inspect whether abnormality of the tablets 4 within the pockets 3 exists or not.

Though temperature measurement by the temperature-measuring section 15 is essential, temperature measurement by the temperature-measuring section 17 is conducted if needed. For example, it is possible to measure temperature by the temperature-measuring section 17, changing speed of temperature is calculated based on a difference between the temperature and that by the temperature-measuring section 15 to inspect whether abnormality of the tablets 4 exists or not. In the case where surface temperature of the blister pack 1 before entering the temperature-varying section 14 is known by other means, temperature measurement by the temperature-measuring section 17 can be omitted.

In measuring surface temperature of the packaging container (e.g., blister pack) having two or more spaces of the invention, temperature of the packaging container at a surface corresponding to each space (pocket) is measured. For identifying the position of the packaging container at the surface corresponding to each space there are two methods: one is based on an optical image of the packaging container (the camera 13 for positional information is used), and the other is based on periodicity of change in the measured temperature. The method identifying the position based on periodicity of change in the measured temperature is as follows: in a case where the individual spaces of the packaging container are arranged in a periodic manner (for example, pockets of a blister pack), measured values of surface temperature of the packaging container during conveyance (for example, measured by the temperature-measuring section 15 in FIG. 1) vary periodically (an example is shown in FIG. 6), accordingly, under the condition where temperature is decreased, a position indicating a maximum in temperature change corresponds to a space where the content is normal, and under the condition where temperature is increased, a position indicating a minimum of temperature change corresponds to a space where the content is normal, so that it is possible to know the position of the surface, of the packaging container, corresponding to each space based on periodicity of temperature change. The method utilizing periodicity in change of surface temperature is more accurate and simpler, and thus, more preferable. Further, in the case of a blister pack, measuring surface temperature on the side of the covering material is preferred to measuring surface temperature on the side of the container film.

Uniformizing surface temperature in the inspection method of the present invention means making difference of surface temperature 3° C. or less among two or more separated spaces of the packaging container where contents are contained next to each other. The difference of surface temperature is preferably 2° C. or less, and more preferably 1° C. or less. For uniformizing surface temperature, a method of leaving the packaging container for a certain period of time under an atmosphere set at a predetermined temperature, a method of blowing air of a predetermined temperature on the packaging container, and a method of bringing the packaging container into contact with a physical body of a predetermined temperature are preferable. The method of blowing air of a predetermined temperature is most preferable, since damage on the packaging container is least and temperature can be uniformized rapidly.

The inspection method of the present invention is suitable as an inspection method of a packaging container in which many contents of a same kind are contained. Since respective spaces to be compared in which contents of the packaging container are contained experience an atmosphere of a same temperature, comparison between a normal portion and an abnormal portion can be performed accurately, which is favorable. Further, in the case where the partitioned spaces (pockets) containing the contents are arranged periodically, identification of the spaces can be made easily based both on an image and on periodicity in temperature change, so that the method is particularly suitable for inspection of contents of a blister pack, in which pockets are arranged periodically.

Surface temperature of a normal spaces (such as the pocket) of the packaging container at a time of temperature measurement is preferably from 0° C. to 50° C., more preferably 10° C. to 45° C., and further preferably 15° C. to 30° C. Change of surface temperature of a normal space of the packaging container before and after the temperature-varying section (14 in FIG. 1) is preferably 0.5° C. to 20° C., more preferably 0.5° C. to 10° C., and further preferably 1° C. to 5° C. If the change of surface temperature of the packaging container is too large, deformation damage is caused due to expansion and contraction of the packaging container, and if the change is too small, inspection accuracy becomes low, which are both unfavorable.

Methods to obtain infrared emittance of a portion to be measured used in measuring surface temperature by an infrared radiation thermometer include (A) calculating emittance from reflectance or absorbance obtained by an infrared spectrometer, (B) using substance-specific data described in documents, (C) adopting infrared emittance that makes a value of surface temperature measured by an infrared radiation thermometer coincide with a value of surface temperature measured by a contact-type surface thermometer. The method (C) is most accurate and thus preferable.

Infrared emittance of a surface for which temperature is to be measured of the packaging container is preferably 0.7 to 1.0, more preferably 0.8 to 1.0, and further preferably 0.9 to 1.0. The closer to 1.0 the infrared emittance is, the more infrared emission light is, which renders surface temperature measurement of the infrared radiation thermometer more accurate, and is favorable.

Infrared emittance of a surface of a packaging container varies according to a kind of the packaging material and a print pattern on the surface (e.g., 6 in FIG. 5). If the kind of the packaging material and the print pattern of a surface to be measured can be known, it is possible to know infrared emittance of that portion. Methods to know a print pattern include knowing it from an optical image of the surface of the packaging container before measuring temperature, and knowing it based on positional information of the packaging container. Difference in infrared emittance between the print pattern of the packaging material (6 in FIG. 5) and an even layer below the pattern (7 in FIG. 5) is preferably 0.4 or less, more preferably 0.2 or less, and further preferably 0.1 or less.

It is also preferable to concurrently use the inspection method of the present invention with an inspection method of a content using an optical image taken after the content is contained in the packaging container but before it is covered with an opaque packaging material (for example, taken by the camera 16 in FIG. 1).

It is possible to input in advance a standard value of change speed of temperature of a space, of the packaging container, whose content is normal, and judge a space (pocket) whose temperature deviates from the standard value by a predetermined value to be abnormal, to make inspection time shorter and to improve inspection accuracy. For the standard value, it is preferable to use a value modified appropriately according to a measured value of surface temperature during manufacture.

Carbon dioxide and moisture in the atmosphere absorb infrared light, and therefore, lower accuracy of temperature measurement by an infrared radiation thermometer. It is preferable that concentration of carbon dioxide and moisture is low in an atmosphere within a temperature-measuring section where an infrared radiation thermometer is used for measuring surface temperature. Concentration of carbon dioxide is preferably 250 ppm or less, more preferably 150 ppm or less, and further preferably 50 ppm or less. Relative humidity is preferably 40 RH % or less, more preferably 30 RH % or less, and further preferably 15 RH % or less.

When surface temperature is measured by an infrared radiation thermometer, infrared light emitted from circumjacent physical bodies lowers accuracy of temperature measurement. By lowering temperature of the circumjacent physical bodies or lowering infrared emittance of the circumjacent physical bodies, infrared radiation light from the circumjacent physical bodies can be reduced to improve accuracy of temperature measurement. Surface temperature of a normal space of the packaging container in the temperature-measuring section is preferably higher than a temperature of an inner wall of the temperature-measuring section of a manufacturing machine by 2° C. or more, and more preferably by 4° C. or more. Materials that have low infrared emittance and thus can be used favorably for an inner wall of a manufacturing machine include stainless steel, aluminum, zinc, tin, copper, titanium, and steel, and a non-oxidized surface thereof is especially preferred. In light of durability, strength, workability and cost, stainless steel is particularly preferable.

Further, accuracy of inspecting whether abnormality of a content exists or not can be improved depending on a configuration and the like regarding to the type of the surface thermometer, measuring condition, adjustment of positional relation between the surface thermometer and the packaging container, adjustment of conveying speed of the packaging container, and the condition of increase in surface temperature or decrease in surface temperature.

### Advantageous Effect of the Invention

According to the inspection method of the present invention, inspecting whether abnormality such as omission, chip, abnormal size, abnormal shape, and contamination exists or not in a content of a packaging container can be performed rapidly, accurate inspection is possible for any combination of a content and a packaging material, an inspection result does not suffer any adverse effect from disturbance, an installation location for inspection equipment is not limited, and quality of a content is not deteriorated by illumination, in inspecting whether abnormality of a content of the packaging container exists or not. Further, inspection can be performed on a packaging container where an opaque packaging material is used, no adverse effect is imposed on pharmaceutical agents, food and the like as the contents unlike an inspection method utilizing X-ray so that a manufacturer can manufacture pharmaceutical agents at ease and at a stable quality, moreover, no reputational damage from disfavoring X-ray is caused, protection of workers against X-ray is not necessary so that inspection can be performed with a small inspection apparatus, and it is possible to perform inspection of abnormality of a content in a packaging container with an inspection apparatus that can be installed easily in existing manufacturing equipment. Furthermore, a discarded amount can be minimized, and inspection of abnormality of a content in a packaging container can be performed under a condition where a corrective action can be taken effectively.

### BRIEF DESCRIPTION OF DRAWINGS

FIG. 1 is a schematic view of a manufacturing machine of a blister pack using an inspection method of a content of a blister pack concerned in a first embodiment of the present invention.

FIG. 2 is a view explaining a general manufacturing method of a blister pack.

FIG. 3 is a perspective view showing a long blister pack before being cut.

FIG. 4 is an enlarged view of a pocket of a blister pack in which a tablet is contained.

FIG. 5 is a view explaining a print pattern.

FIG. 6(a) is a cross-sectional view of a blister pack at a plane parallel to a conveying direction, and (b) is a view explaining a change of surface temperature corresponding to positions of (a).

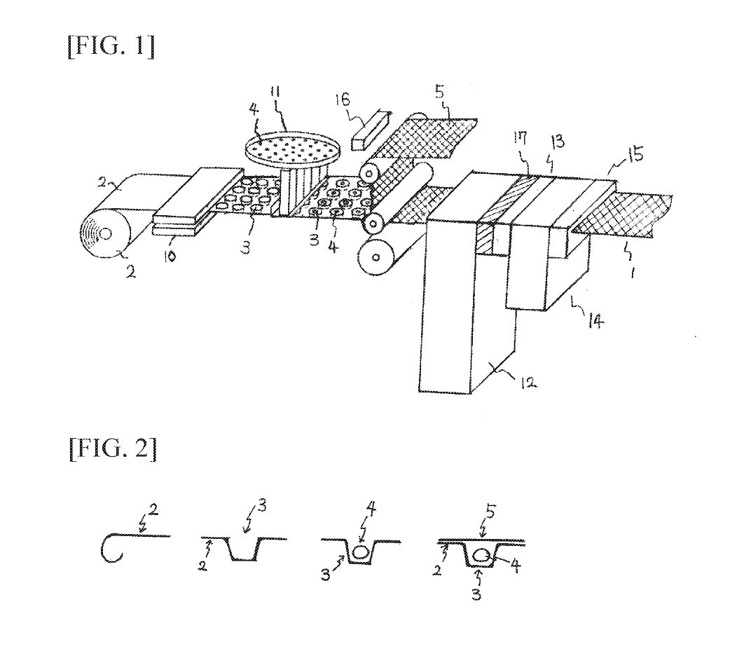

### DETAILED DESCRIPTION OF PREFERRED EMBODIMENTS

Hereafter, the present invention will be described in detail based on examples.

### EXAMPLE 1<Preparation and inspection of blister pack A>

A container film 2 (width: 70 mm, length: 3 m, film thickness: 220 pm) in which aluminum foil is attached to a polyvinyl chloride film is arranged such that the aluminum foil is to be an inner portion of a pocket, and pockets 3 (diameter: 10 mm, depth: 3.3 mm, interval between centers of the pockets in both longitudinal and lateral directions: 15 mm) were formed in four rows in the lateral direction by a heating-sucking processing machine 10. Tablets 4 of vitamin C (diameter: 9 mm, height: 3 mm) are scattered onto the container film so as to be contained in the pockets 3, and a covering material 5 (width: 70 mm, length 3 m, film thickness 40 μm), which was made of aluminum foil and a film made of a vinyl chloride film to which a layer containing fine particles of titanium oxide was attached, was arranged such that a side of the aluminum foil is in contact with a periphery of an opening of the pocket, and attached so as to cover the opening of the pocket 3. Note that, as the tablets 4, normal products and abnormal products chipped half were used, and no tablet 4 was contained in some of the pockets 3. Air of 27° C. was blown onto the blister pack A in a temperature-uniformizing section 12 (length: 2 m). Surface temperature of the blister pack A at the exit of the temperature-uniformizing section 12 was within a range between 26 and 27° C. Subsequently, air of 20° C. was blown in a temperature-varying section 14 (length: 40 cm), and surface temperature was measured from a side of the covering material 5 in a temperature-measuring section 15 (length: 10 cm) using a commercially available infrared radiation thermometer with infrared emittance being set to 0.91. Temperature of an inner wall (stainless steel) of the temperature-measuring section 15 was 20° C. Conveying speed was 2 m/min. The result of measurement of surface temperature is an average value of measurements at five pockets of each of the normal and abnormal (half chip, omission) portions. Further, it was confirmed that surface temperature changed periodically due to conveyance of blister pack A and that a location that showed a maximum value of surface temperature coincided with the center of the pocket.

### <Result of Measurement of Surface Temperature>

(a) Pocket where a tablet 4 was contained normally (normal): 25.4° C.

(b) Pocket where a tablet 4 chipped half was contained (abnormal): 23.0° C.

(c) Pocket where a tablet 4 was omitted (abnormal): 21.5° C.

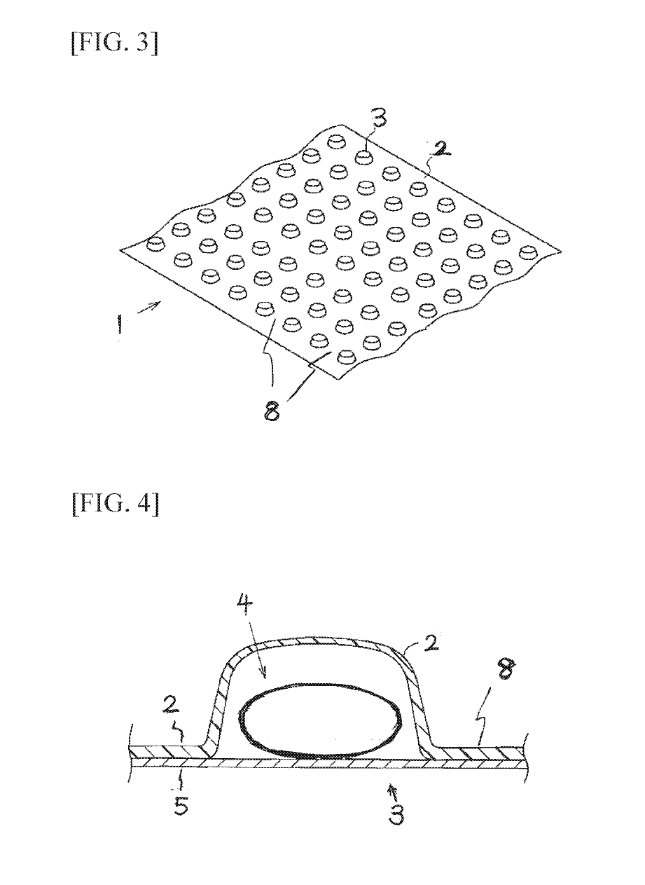

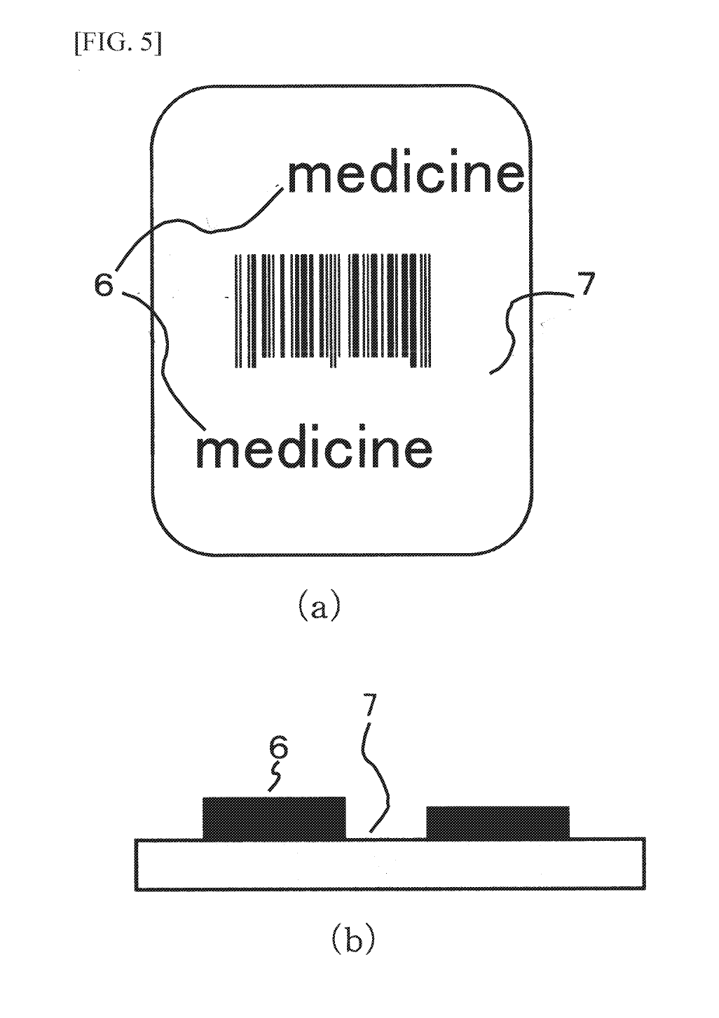

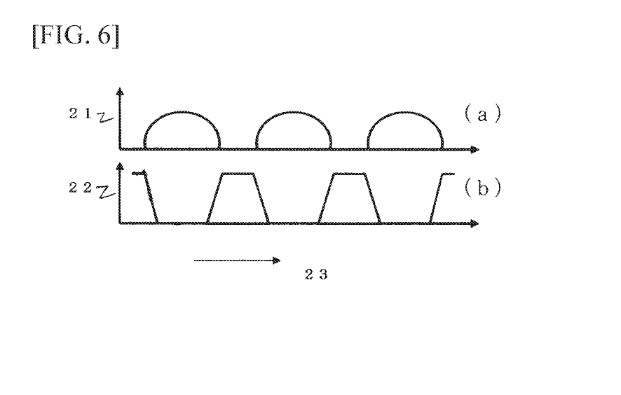

(x) Location between pockets (8 in FIGS. 3 and 4): 21.6° C.

Surface temperature of both of the abnormal portion (b) where the tablet was chipped half and the abnormal portion (c) where a tablet was omitted was lower than that of the normal portion (a) where the tablet was normal. Abnormality of omission and half chip of a tablet could be inspected by the method using a difference in temperature-decreasing speed of the present invention.

### EXAMPLE 2

### <Preparation and Inspection of Blister Pack B>

Blister pack B was prepared and inspected in a similar manner to blister pack A of Example 1 except that air of 20° C. was blown in the temperature-uniformizing section 12, air of 27° C. was blown in the temperature-varying section 14 and temperature of the inner wall of the temperature-measuring section 15 was set to 18° C. Note that surface temperature of blister pack B at the exit of the temperature-uniformizing section 12 was within a range between 20 and 21° C. Further, it was confirmed that surface temperature changed periodically due to conveyance of blister pack B and that a location that showed a minimum value of surface temperature coincided with the center of the pocket.

### <Result of Measurement of Surface Temperature>

(d) Pocket where a tablet 4 was contained normally (normal): 21.6° C.

(e) Pocket where a tablet 4 chipped half was contained (abnormal): 23.2° C.

(f) Pocket where a tablet 4 was omitted (abnormal): 24.9° C.

(y) Location between pockets (8 in FIGS. 3 and 4): 25.3° C.

Surface temperature of both of the abnormal portion (e) where the tablet was chipped half and the abnormal portion (f) where a tablet was omitted was higher than that of the normal portion (d) where the tablet was normal. Abnormality of omission and half chip of a tablet could be inspected by the method using a difference in temperature-increasing speed of the present invention.

### INDUSTRIAL APPLICABILITY

The inspection method of the present invention can be applied to inspection of products while being manufactured in pharmaceutical industry, food manufacturing industry, and the like. Particularly, the inspection method can be suitably employed for inspection of a packaging container during manufacturing of a product of the form of a blister pack (including a press through pack (PTP)) that contains tablets, capsules, ampules or jelly of pharmaceutical agents or food.

### DESCRIPTION OF REFERENCE NUMERALS

1: blister pack, 2: container film, 3: pocket, 4: tablet, 5: covering material, 6: print pattern, 7: uniform surface of container film or covering material, 8: location between pockets, 10: heating-sucking processing machine, 11: tablet-feeding hopper, 12: temperature-uniformizing section, 13: camera for positional information, 14: temperature-varying section, 15: temperature-measuring section, 16: camera for image inspection, 17: temperature-measuring section, 21: height, 22: temperature, 23: conveyance direction

1. An inspection method of a content in a packaging container, the inspection method comprising: inspecting abnormality in the content by employing either a difference in increasing speed of temperature or a difference in decreasing speed of temperature of the packaging container.

2. The inspection method of the content in the packaging container of claim 1, wherein the packaging container has two or more spaces separated by partitions, a content of a same type is contained in each of the spaces, and temperature of the packaging container corresponding to each of the spaces is measured.

3. The inspection method of the content in the packaging container of claim 2, wherein the packaging container is a blister pack including a belt-shaped container film having a pocket formed therein in which the content is contained, and a belt-shaped covering material which is adhered to the container film so as to cover the pocket.

4. The inspection method of the content in the packaging container according to claims claim 1, wherein temperature of the packaging container is made uniform before temperature of the packaging container is increased or decreased.
